(19) United States
(12) Patent Application Publication
(10) Pub. No.: US 2016/0067076 A1
(43) Pub. Date: Mar. 10, 2016

(54) Apparatus to Eliminate Mobility Pain in Individuals with Walking Infirmities

(71) Applicant: Donald Arthur Budeit, Canton, GA (US)

(72) Inventor: Donald Arthur Budeit, Canton, GA (US)

(21) Appl. No.: 14/476,876

(22) Filed: Sep. 4, 2014

(51) Int. Cl. A61F 5/01; A61H 3/00

(52) U.S. Cl. CPC A61F 5/0123; A61H 3/00

(57) ABSTRACT

This is an apparatus for eliminating pain associated with walking in individuals with knee, lower leg or foot infirmities. The apparatus is comprised of a specially designed cane (14) attached to a brace (10) that encircles the upper leg in the thigh area. The cane freely passes through a positioning loop (36) strapped in the ankle area and extends a couple inches below the arch of the wearers foot. There is a self-locking knuckle joint (30) in the cane at the location of the wearers knee that bends in conformity with it to enable walking with a normal gait. The knuckle joint is designed to be enclosed and self-locked within the upper portion of the cane when weight is put upon the base of the cane and retracted when the leg is lifted. 100% of body weight is off-loaded to the thigh area.

### BACKGROUND

1. Field of the Invention

The present invention is an apparatus for eliminating pain associated with walking in individuals with knee, lower leg, or foot infirmities

2. Description of Prior Art

There are many different types of devices to aid in mobility of affected persons. There are disadvantages to all of them.

Knee Braces—This is an apparatus with straps above the ankle and above the knee and may be hinged at the knee. These devices may lessen the pain in some individuals but not totally eliminate the pain when weight is put upon the foot as in walking or standing.

Knee Walkers—These devices are cumbersome wheeled vehicles that cradle the knee to take the weight off an injured foot or ankle and are propelled by the uninjured foot. The knee is still carrying all the body weight.

Crutches—These are devices that extend the length of the body , are cradled under the arm pit or pits and are normally used when it is necessary to take all of the weight off of an injured extremity such as a foot, ankle, knee or hip. Climbing stairs cannot be safely accomplished.

Knee Crutch—This device is used in lieu of crutches. Body weight is still upon the knee.

Freedom Leg—This devise transfers body weight directly to the thigh area but the design is such that the wearers knee is held in one position and creates a hobble in walking rather than a normal gait.

Stance Control Knee Joint device—These devices have knee joints that are mechanical or micro processer controlled and do not take the pressures off an injured foot or ankle.

In summation, there are disadvantages to all of these prior art devices for total elimination of pain associated with walking.

### OBJECTS ANS ADVANTAGES OF THE INVENTION

The present invention overcomes all of the disadvantages of prior art devices. The primary function of the apparatus is to eliminate the pressures on an injured foot, ankle, lower leg and or knee when weight is put upon the foot as in walking and or standing. The apparatus consists of a novel cane attached to a brace worn in the area of the thigh and that has a self locking joint that bends in conformity with wearers knee joint and offloads 100% of body weight directly from the base of the cane to the thigh area thereby eliminating pain from walking.

### ADDITIONAL ADVANTAGES OVER PRIOR ART

The present invention in most cases is worn beneath clothing and is not obvious. Only the inch or so of the cane protrudes below clothing. Walking with a normal gait is possible and gets no unwanted attention. When sitting down at a table or in a booth the knee is bent in a normal position. Nothing is removed to be stacked against a wall or table. When climbing stairs, both hands are free to hold onto hand rails.

### ADDITIONAL ADVANTAGES OVER PRIOR ART

Braces cannot totally eliminate pain associated with mobility. In addition, braces are useless for most foot or lower leg injuries.

Knee Walkers are cumbersome, have to be carted around, are obvious and normally require solid footing for ease of use.

Crutches are obvious, have to be carted around and place against adjacent tables or walls when not in use.

Freedom Leg is a device like crutches that takes weight off an injured foot. It has composit bars with a permanent angle that creates a hobble when you walk.

Stance Control Knee Joint devices cradle the foot and are useless for foot injuries.

### OBJECTS OF THE PRESENT INVENTION

The primary object of the apparatus is to eliminate 100% of the pain associated with walking in individuals with foot, ankle lower leg and or knee injuries or deterioration.

Another object is to walk with a normal gait.

Another object is to be able to sit in in a chair with legs bent in a normal position.

Another object is to retain the ability to climb stairs safely with both hands free to hold onto hand rails.

Another object is to eliminate the need for excess baggage that draws attention to your infirmity.

Another object is to enable entering, exiting, and operating automobiles without off loading mobility equipment.

It should be apparent from all the forgoing that this apparatus is a novel invention and has numerous advantages over prior art and eliminates the disadvantages. It is not a Specialized device designed for one specific mobility infirmity. It is a catch all apparatus that can be used in lieu of or replace almost all prior art devices to enable normal gait mobility without pain for injuries or infirmities from the thigh down to the bottom of the feet.

### BRIEF DESCRIPTION OF THE DRAWING FIGURES

FIG. 1. Overall view of the apparatus

Brace with connecter and three section cane that freely passes through a Positioning

Loop

FIG. 2. Inside view

Cane 14A and 14B with knuckle joint self-locked within Cane 14A

FIG. 3. Inside view

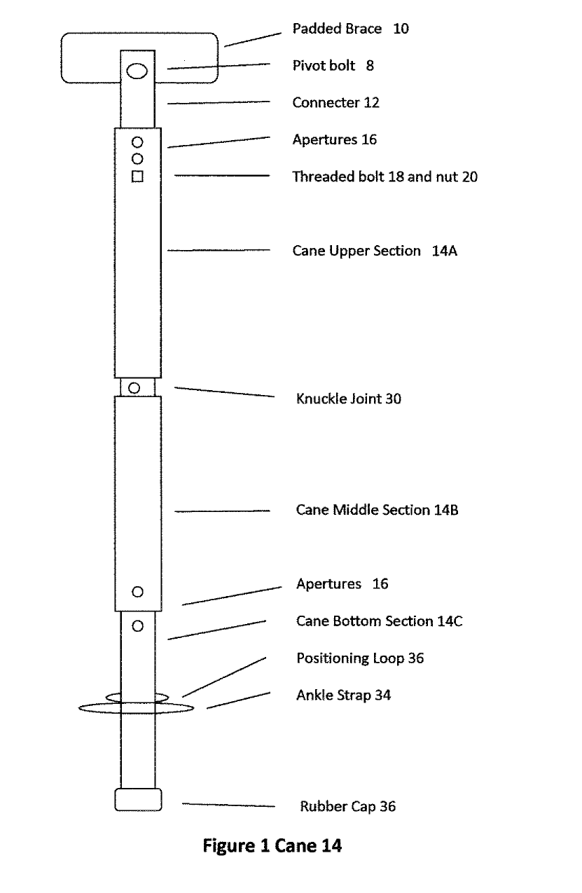

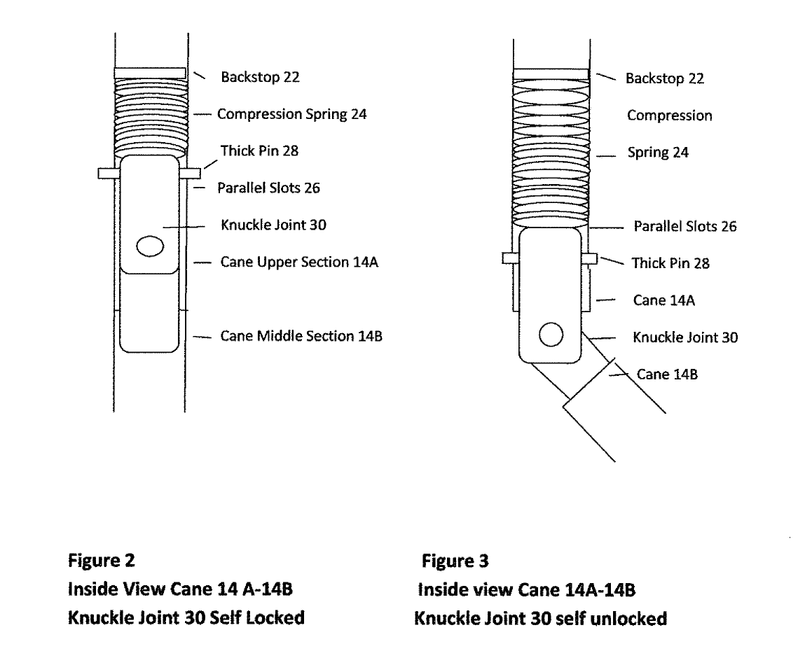

Cane 14A and 14B with knuckle joint self-unlocked.

### DETAILED DESCRIPTION OF THE INVENTION

This is an apparatus that eliminates body weight on an injured or deteriorated foot, ankle, lower leg, and or knee by offloading the weight to an area above the knee, thereby eliminating any pain from walking and or standing on the damaged extremity.

The apparatus is comprised of an adjustable padded brace (10) wrapped around the upper leg in the area of the thigh. Attached to the brace by a pivot bolt (8) is a connecter (12) comprised of hollow metallic tubing which is inserted into a cane upper section (14A) of a hollow metallic three section cane (14). The connecter (12) and cane upper section (14A) each have a series of apertures (16) on both sides and are aligned with each other to extend the length of cane upper section (14A). A threaded bolt (18) and a nut (20) in two aligned apertures extend the two sections. The cane upper section (14A) contains a compression spring backstop (22) and a compression spring (24). At the base of upper section (14A) is the upper half of a knuckle joint (30A). A thick pin (28) in knuckle joint upper half (30A) projects through parallel slots (26) on each side of cane upper section (14A). The slots (26) are approximately one inch long. The knuckle joint lower half (30B) is fused into the top of cane middle section (14B).The cane bottom section (14C) has a lessor diameter than cane middle section (14B) and is inserted into cane middle section (14B). Cane middle section (14B) and cane bottom section (14C) each have a series of apertures (16) on both sides. A threaded bolt (18) and a nut (20) in two aligned apertures (16) extend the length of cane bottom section (14C). Cane bottom section (14C) freely passes through a positioning loop (36) attached to a adjustable strap (34) located in the wearers ankle area. At the base of the cane is a rubber cap (36). When weight is put upon the base of the cane (14) as with a normal walking gait, the upper half knuckle joint (30A) in cane upper section (14A) compresses the compression spring (22). The maximum insertion of knuckle joint (30) is when thick pin (28) contacts the end of the slots (26). At this point, the joinof knuckle joint (30) is totally enclosed within cane upper section (14A) and body weight is transferred from the lower extremities to the thigh area. The cane (14) is completely rigid until weight is taken off the base of the cane as by lifting the damaged extremity as with a normal walking gait.

Pivot bole (18) and positioning loop (36) keeps ca ne (14) aligned with the center of the arch of the wearers foot. Adjusting the length of cane upper section (14A) lines up Knuckle joint (30) and the wearers knee to bend in conformity with each other. Adjusting the length of cane bottom section (14C) is to have a proper distance from the canes base to the bottom of a shoe or bandaged foot.

### REFERENCE NUMBER LISTING

8 Pivot Bolt
10 Padded Brace
12 Connecter
14 Cane
14A Cane Upper Section
14B Cane Middle Section
14C Cane Bottom Section
16 Apertures
18 Threaded Bolt
20 Nut
22 Compression Spring Backstop
24 Compression Spring
26 Parallel Slots
28 Thick Pin
30 Knuckle Joint
30A Knuckle Joint Upper Half
30B Knuckle Joint Lower Half
34 Ankle Strap
36 Positioning Loop
38 Rubber Cap

### OPERATION OF THE INVENTION

The primary object of this apparatus is to enable individuals with lower leg infirmities to walk with a normal gait without pain.

This is accomplished by using a brace (10) that encircles the leg above the knee. Attached to the brace by a connecter (12) is a three section cane (14) that that extends downward and has a knuckle joint (30) in the cane at the location of the wearers knee joint. The upper section of the cane (14A) contains a compression spring backstop (22) and a compression spring (24) which when compressed allows the joint to be self-locked within the cane upper section (14A). The bottom section of the cane (14C) freely passes through a positioning loop (36) attached to a strap (34) located just above the ankle. This keeps the base of the cane (14) positioned near the center of the arch of the foot. The cane (14) extends one inch or more below whatever shoe is worn on the foot or below any bandage on a shoeless foot. In a normal walking gait, when an injured foot is brought forward, weight on the base of the cane (14) compresses the spring (24) about one half inch self-locking the knuckle joint (30) to keep the cane rigid . As the injured leg is lifted off the ground, the spring (24) decompresses and permits bending of the joint (30). As weight is put on the base of the cane (14), 100% of the weight is transferred to the upper portion of the brace (10), which eliminates any weight upon the injured foot, ankle or knee, thereby eliminating any pain associated with walking and or standing.

Use of the apparatus is simple. Strap the brace with cane attached to the upper leg in the area of the thigh. Attach the ankle strap with the positioning loop around the cane. Walk away—Walk away from misery—Walk away from pain—Walk away with a spring in your step, a smile on your face and a song in your heart—Walk away

### CONCLUSION

It should be apparent that the apparatus is a novel invention that overcomes all the disadvantages of prior art devices.

It totally eliminates the pressures on an injured foot, ankle, lower leg and or knee when weight is put the foot as in walking and or standing.

It offloads 100% of body weight directly from the base of the cane to the thigh area thereby eliminating all pain from walking and or standing.

In most cases, it can be worn beneath clothing and is not obvious and gets no unwanted attention. Only the inch or so of cane protrudes below clothing.

The novel self-locking knuckle joint enables injured individuals with lower leg infirmities to walk with a normal gait.

Climbing stairs presents no problem. Both hands are free to hold onto hand rails.

The apparatus enables sitting in a chair or booth or at a table with legs bent in a normal position.

Nothing has to be removed to enter, exit or drive vehicles.

Nothing has to be removed and stacked against a wall in a restaurant or a place of business.

It has a small footprint and is light in weight.

From an operational standpoint, the apparatus stands alone by enabling mobility without pain in individuals with foot, ankle, knee and lower leg infirmities. The novel self locking knuckle joint is fail safe. It has no mechanical or microprocessor controlled attachments. It self-locks when weight is put upon the base of the cane and self-unlocks when the leg is lifted as with a normal walking gait. The pivot bolt connection of the cane to the brace coupled with the positioning loop maintains alignment with the center of the arch of the wearers foot.

From a convenience and marketing standpoint, the apparatus stands alone. The adjustments in length from the brace to the knuckle joint and from the knuckle joint to the base of the cane enables one adult size fit most adults and be an off the shelf apparatus.

From all the forgoing, it should be apparent that this apparatus that has a small footprint is a giant step forward in the elimination of mobility pain in individuals with foot, ankle, lower leg and or knee infirmities.

None of the prior art devices enable their owners to do all of the things this apparatus would enable them to do.

The apparatus enables individuals with lower leg infirmities to lead a normal life, to go where ever they want to go and do whatever they want to do.

Best of all, this apparatus enables its wearer to Walk Away—Walk Away from Misery—Walk Away from pain—Walk Away with a spring in their step, a smile on their face and a song in their hearts—Walk Away.

### RAMIFICATIONS AND SCOPE OF THE INVENTION

While my apparatus description contains many specifics, they should not be construed as limitations on the scope of the invention but rather as an exemplification of one preferred embodiment thereof. Many other variations are possible. For example, the knuckle joint (30) can be replaced by other types of joints. The bolt through apertures (16) to lengthen sections could be replaced by the button and spring or other methods. These and other changes have been contemplated by the inventor and fall within the scope of the invention. Accordingly, the scope of the invention should be determined not by the embodiment illustrated, but by the appended claims and their legal equivalents.

I claim:

1. An apparatus designed to facilitate mobility in individuals with foot, ankle, lower leg and or knee injuries or deterioration, said apparatus being the means to walk with a normal gait without pain, said apparatus comprised of a brace, said brace coupled with a connector held in place by a pivot bolt, said connector attached to a cane, said cane comprised of a upper section, a middle section, and a bottom section, said upper section extending downwardly said upper section having a means to extend its length, said upper section having a compression spring encased within it, the base of said compression spring in contact with a knuckle joint, said knuckle joint attached to said middle section, said knuckle joint becoming encased within said upper section when body weight from normal gait walking compresses said compression spring, said knuckle joint containing a thick pin that projects through a pair of parallel slots in said upper section, said thick pin and said parallel slots being the means to limit the penetration and encasement of said knuckle joint, to be the means to limit the retraction of said knuckle joint, and to be the means of maintaining the alignment of said upper section with said middle section, said middle section attached to said bottom section, said bottom section having means to increase its length, said bottom section extending through a positioning loop attached to a strap located just above an ankle, said positioning loop and said pivot bolt being the means to keep said cane positioned near the center of a foots arch, said bottom section of said cane adjusted to a length approximately one inch below the sole of a shoe or bandaged foot.
